SEQUENCE LISTING (14 sequences)

SEQ ID NO: 1 - DNA, 21 bp, Artificial sequence
  synthetic DNA
gtgatagatttcttttgcaat

SEQ ID NO: 2 - DNA, 21 bp, Artificial sequence
  synthetic DNA
accctattcatcctctgctcc

SEQ ID NO: 3 - DNA, 27 bp, Artificial sequence
  synthetic DNA
ttccaggagggtttcgaacggaccata

SEQ ID NO: 4 - DNA, 24 bp, Artificial sequence
  synthetic DNA
ctagaggtgcaacgaataaaactt

SEQ ID NO: 5 - DNA, 27 bp, Artificial sequence
  synthetic DNA
tatggtccgttcgaaaccctcctggaa

SEQ ID NO: 6 - DNA, 20 bp, Artificial sequence
  synthetic DNA
aggatcctgacctggaaaca

SEQ ID NO: 7 - DNA, 1365 bp, Veronica persica
  CDS (1)..(1365)
atggaagacacaatcatcctctatgcttcatccgtgcacctgaactctgtgctagtaatagccaagttcataaacaaacatcatccttctatctccataatcatactcagcaatgctcctgattcagccgcatcttccattacctctgaagcctcatcaatcacttaccatcgactccctactcccgacattcctcccaacatcatcactaatccagtcgaacttcttttcgaggttccacgcctcaacaatcccaatgtcaaacaataccttgaacaaatctcccaaaaaactaatgtcaaagcattcatcattgatttcttttgcaactcagcttttgaagtttctacgagtttgaacattccaacctacttctacgtcagcagtggcggtttcggcctctgtgctttcctccacttcccaaccacggacgaaatcatccctcaagatatcggagacttgaacgattatctggaaatcccaggctgcccacccgttcactctttagatttccctaaaggaatgtttttcaggcacactaatacccacaatcatttccttgacactgccagaaacatgaggaaagccaatgggattctggtgaactcgttcgatgctcttgagtatagatctaaagcagctttattgaacggaatttgcgttccgaacggtccaacaccccaagttttattcgttgcacctctagttactggaatgaacagtagaaaaggcgactcggagcatgaatgtttaagctggcttgactcacaaccaagtaagagtgtaattttcctatgttttggcagaaagggttttttctccaaacaacagttgcaagaaatagcaactggcttggaaaacagtggccataggtttctatggtccgttcgaaaccctcctggaattaataatgaggatcctgacctggaaacacttcttccagagggttttctggaaaggactaaagaacgaggattcgtgataaagtcatgggcgcctcagaaagaagtactaagccatgagtccgttggagggttcgtgacacattgtggtaggagttcgatattagaagcagtgtcattcggtgtgcctatgattggttttccaatatacgcggagcaaaggatgaatcgggtattcatggttgaggaaatgaaagtgtcattgccgttagatgaggctggtgatggacttgttacgtccggtgagctcgaaaagcgagtgaaggaattgatgggttcggttagtgggaaagcgattcgacaacgagttaatgagttgaaagtttcgggcgaggcagcggtgaaggaaggtggttcttcagtggttgatctggacaagttcatcaagttggttattgggtaa

SEQ ID NO: 8 - protein, 454 aa, Veronica persica
MEDTIILYASSVHLNSVLVIAKFINKHHPSISIIILSNAPDSAASSITSEASSITYHRLPTPDIPPNIITNPVELLFEVPRLNNPNVKQYLEQISQKTNVKAFIIDFFCNSAFEVSTSLNIPTYFYVSSGGFGLCAFLHFPTTDEIIPQDIGDLNDYLEIPGCPPVHSLDFPKGMFFRHTNTHNHFLDTARNMRKANGILVNSFDALEYRSKAALLNGICVPNGPTPQVLFVAPLVTGMNSRKGDSEHECLSWLDSQPSKSVIFLCFGRKGFFSKQQLQEIATGLENSGHRFLWSVRNPPGINNEDPDLETLLPEGFLERTKERGFVIKSWAPQKEVLSHESVGGFVTHCGRSSILEAVSFGVPMIGFPIYAEQRMNRVFMVEEMKVSLPLDEAGDGLVTSGELEKRVKELMGSVSGKAIRQRVNELKVSGEAAVKEGGSSVVDLDKFIKLVIG

SEQ ID NO: 9 - DNA, 27 bp, Artificial sequence
  synthetic DNA
cacccatatggaagacacaatcatcct

SEQ ID NO: 10 - DNA, 30 bp, Artificial sequence
  synthetic DNA
ctcgagtttttacccaataaccaacttgat

SEQ ID NO: 11 - DNA, 17 bp, Artificial sequence
  synthetic DNA
gcggtttcggcctctgt

SEQ ID NO: 12 - DNA, 24 bp, Artificial sequence
  synthetic DNA
tccgatatcttgagggatgatttc

SEQ ID NO: 13 - DNA, 20 bp, Artificial sequence
  synthetic DNA
gcggaaggatcattgtcgat

SEQ ID NO: 14 - DNA, 20 bp, Artificial sequence
  synthetic DNA
ctagcgggcggagcttatta

(19) United States
(12) Patent Application Publication
(10) Pub. No.: US 2011/0219476 A1
(43) Pub. Date: Sep. 8, 2011

(54) GLUCURONYL TRANSFERASE AND POLYNUCLEOTIDE ENCODING THE SAME

(76) Inventors: Eiichiro Ono, Osaka (JP); Yuko Fukui, Osaka (JP)

(21) Appl. No.: 13/062,093

(22) PCT Filed: Sep. 4, 2008
(86) PCT No.: PCT/JP2008/066365

§ 371 Date: May 17, 2011

(51) Int. Cl. A01H 5/00; C12N 15/63; C12N 5/10; C12N 1/00; C12P 19/44; C07H 21/04; C12N 9/10

(52) U.S. Cl. 800/298; 435/320.1; 435/419; 435/325; 435/243; 435/74; 536/23.2; 435/193

(57) ABSTRACT

The present invention provides a novel glucuronosyltransferase, a polynucleotide encoding the same (e.g., a polynucleotide comprising a polynucleotide consisting of the nucleotide sequence at positions 1 to 1362 in the nucleotide sequence represented by SEQ ID NO: 7, or a polynucleotide comprising a polynucleotide encoding a protein having the amino acid sequence represented by SEQ ID NO: 8); and so on. A novel glucuronosyltransferase having a broad substrate specificity and others can thus be provided.

### TECHNICAL FIELD

The present invention relates to a glucuronosyltransferase, a polynucleotide encoding the same, a vector comprising the same, a transformant, and so on.

### BACKGROUND ART

Flavonoids are a collective term for plant secondary metabolites in the phenylpropanoid pathway. Anthocyanins, one type of the flavonoids, are major color pigments which determine flower colors of especially red or orange to bluish purple. Flavone or flavonol glycosides, which are also one type of the flavonoids, themselves display a pale yellow color but form complexes with anthocyanin pigments to exert great effects on the color hue of flowers and are therefore called copigments. In general, a shift of flower color toward the blue wavelength side is called copigmentation.

Apigenin 7-O-glucuronide (glucuronide conjugate, also called as glucuronide glycoside) is accumulated in the petals of snapdragon or Lamiales, Scrophulariacea, Antirrhinum majus, which is considered to function as a copigment (Document 1: Asen, S. et al., Phytochemistry 11, 2739-2741, 1972). The pigment from the blue petals of Asterales, Asteraceae, Centaurea cyanus forms a metal complex and flavone 7-O-glucuronides are found also in the metal complex (Document 2: Shiono, M. et al., Nature 436, 791-792, 2005).

Flavone 7-O-glucuronides called baicalin having an anti-inflammatory activity are accumulated in the radix of Lamiales, Lamiaceae, Scutellaria baicalensis. The radix is used as a Chinese medicinal herb (owgon) exhibiting stomachic effects (Document 3: Gao, Z. et al., Biochemica et Biophysica Acta 1472, 643-650, 1999). SbUBGAT (also called as Sb7GAT) is purified from the radix of Scutellaria baicalensis as a transferase which transfers glucuronic acid to the 7-O-position of baicalein (Document 4: Nagashima S. et al., Phytochemistry 53, 533-538, 2000). The gene corresponding to the Sb7GAT has been registered in GenBank (Accession No. AB042277) but its function remains unidentified. In recent years, it has become clear that 7-O-glucuronides of various flavones are accumulated also in the leaves of Lainiaceae Perilla frutescens that are routinely eaten, and their functionality in human health is expected (Document 5: Yamazaki, M. et al. Phytochemistry 62, 987-998, 2003).

As such, flavone 7-O-glucuronides are plant secondary metabolites which draw much attention in the field of flower color and health food. In spite, their biosynthetic enzymes (e.g., glucuronosyltransferases) remain poorly understood.

### DOCUMENTS

1. Asen, S. et al., Phytochemistry 11, 2739-2741, 1972
2. Shiono, M. et al., Nature 436, 791-792, 2005
3. Gao, Z. et al., Biochemica et Biophysica Acta 1472, 643-650, 1999
4. Nagashima S. et al., Phytochemistry 53, 533-538, 2000
5. Yamazaki, M. et al., Phytochemistry 62, 987-998, 2003

### DISCLOSURE OF INVENTION

Under these circumstances, it has been desired to identify a novel glucuronosyltransferase having a broader substrate specificity and a gene encoding the same.

In view of the foregoing circumstances the present invention has been made and provides the following glucuronosyltransferases and polynucleotides encoding the same, as well as vectors comprising the same, transformants, and so on.

(1) A polynucleotide of any one of (a) to (f) below:

(a) a polynucleotide comprising a polynucleotide consisting of a nucleotide sequence at positions 1 to 1362 in the nucleotide sequence represented by SEQ ID NO: 7;

(b) a polynucleotide comprising a polynucleotide encoding a protein having the amino acid sequence represented by SEQ ID NO: 8;

(c) a polynucleotide comprising a polynucleotide encoding a protein consisting of the amino acid sequence represented by SEQ ID NO: 8 in which 1 to 15 amino acids are deleted, substituted, inserted and/or added and having a UDP-glucuronosyltransferase activity;

(d) a polynucleotide comprising a polynucleotide encoding a protein having an amino acid sequence having at least 80% homology to the amino acid sequence represented by SEQ ID NO: 8 and having a UDP-glucuronosyltransferase activity;

(e) a polynucleotide comprising a polynucleotide that hybridizes under stringent conditions with a polynucleotide consisting of a nucleotide sequence complementary to the nucleotide sequence at positions 1 to 1362 in the nucleotide sequence represented by SEQ ID NO: 7 and encodes a protein having a UDP-glucuronosyltransferase activity; and,

(f) a polynucleotide comprising a polynucleotide that hybridizes under stringent conditions with a polynucleotide consisting of a nucleotide sequence complementary to a nucleotide sequence of a polynucleotide encoding a protein consisting of the amino acid sequence represented by SEQ ID NO: 8, and encodes a protein having a UDP-glucuronosyltransferase activity.

(2) The polynucleotide according to claim 1, which is any one of (g) to (j) below:

(g) a polynucleotide comprising a polynucleotide encoding a protein consisting of the amino acid sequence represented by SEQ ID NO: 8 in which at most 10 amino acids are deleted, substituted, inserted and/or added and having a UDP-glucuronosyltransferase activity;

(h) a polynucleotide comprising a polynucleotide encoding a protein having an amino acid sequence having at least 90% homology to the amino acid sequence represented by SEQ ID NO: 8 and having a UDP-glucuronosyltransferase activity;

(i) a polynucleotide comprising a polynucleotide that hybridizes under high stringent conditions with a polynucleotide consisting of a nucleotide sequence complementary to the nucleotide sequence at positions 1 to 1362 in the nucleotide sequence represented by SEQ ID NO: 7 and encodes a protein having a UDP-glucuronosyltransferase activity; and,

(j) a polynucleotide comprising a polynucleotide that hybridizes under high stringent conditions with a polynucleotide consisting of a nucleotide sequence complementary to a nucleotide sequence of a polynucleotide encoding a protein consisting of the amino acid sequence represented by SEQ ID NO: 8 and encodes a protein having a UDP-glucuronosyltransferase activity.

(3) The polynucleotide according to (1) above, which comprises a polynucleotide consisting of the nucleotide sequence at positions 1 to 1362 in the nucleotide sequence represented by SEQ ID NO: 7.

(4) The polynucleotide according to (1) above, which comprises a polynucleotide encoding a protein consisting of the amino acid sequence represented by SEQ ID NO: 8.

(5) The polynucleotide according to any one of (1) to (3) above, which is a DNA.

(6) A protein encoded by the polynucleotide according to any one of (1) to (5) above.

(7) A vector comprising the polynucleotide according to any one of (1) to (5) above.

(8) A transformant transformed with the polynucleotide according to any one of (1) to (5) above.

(9) A transformant transformed with the vector according to (7) above.

(10) A method for producing the protein of (6) above, which comprises using the transformant according to (8) or (9) above.

(11) A method for producing a glucuronide conjugate, which comprises forming the glucuronide conjugate from UDP-glucuronic acid and a glycosyl acceptor substrate using the protein according to (6) above as a catalyst.

The polynucleotide of the present invention is useful, for example, for the production of a novel glucuronosyltransferase which comprises introducing the polynucleotide into a transformant. In a preferred embodiment of the present invention, the glucuronosyltransferase has a broader substrate specificity and an activity for glucuronidation of various glycosyl acceptor substrates.

### BRIEF DESCRIPTION OF DRAWINGS

FIG. 1 is a photograph of SDS-PAGE in which Escherichia coli expression protein was confirmed. M: size marker, P: pellet fraction, C: crude enzyme fraction, FT: non-adsorbed fraction, 50: fraction eluted with 50 mM imidazole, 200: fraction eluted with 200 mM imidazole, 500: fraction eluted with 500 mM imidazole, arrow: target protein (VpF7GAT) in which Escherichia coli was expressed.

FIG. 2(A) is a chart showing the results of HPLC analysis of apigenin. FIG. 2(B) is a chart showing the results of HPLC analysis of the enzyme reaction solution (apigenin and UDP-glucuronic acid). FIG. 2(C) is a chart showing the results of HPLC analysis of apigenin 7-O-glucuronide. FIG. 2(D) is a chart showing the measurement results of the enzyme reaction solution (apigenin and UDP-glucuronic acid) by TOF-MS.

FIG. 3 is a graph showing the analysis results of the glycosyl acceptor substrate of VpF7GAT for its substrate specificity.

FIG. 4 is a graph showing the expression analysis of the VpF7GAT gene in different organs by quantitative RT-PCR.

### [Sequence Listing Free Text]

SEQ ID NO: 1: synthetic DNA

SEQ ID NO: 2: synthetic DNA

SEQ ID NO: 3: synthetic DNA

SEQ ID NO: 4: synthetic DNA

SEQ ID NO: 5: synthetic DNA

SEQ ID NO: 6: synthetic DNA

SEQ ID NO: 9: synthetic DNA

SEQ ID NO: 10: synthetic DNA

SEQ ID NO: 11: synthetic DNA

SEQ ID NO: 12: synthetic DNA

SEQ ID NO: 13: synthetic DNA

SEQ ID NO: 14: synthetic DNA

### BEST MODE FOR CARRYING OUT THE INVENTION

Hereinafter, the glucuronosyltransferases of the present invention, the polynucleotide encoding the same, the vector comprising the same, the transformant and so on are described in detail.

The main anthocyanin pigment of V. persica belonging to the genus Veronica of the family Scrophulariaceae in the order Lamiales, which produces a blue flower, is deiphinidin or 3-O-(3-O-(6-O-coumaroyl)-glucosyl)-6-O-coumaroyl-glucoside-5-O-glucoside and the main flavone is apigenin 7-O-(3-O-glucuronosyl)-glucuronide (Miho Ruike, Master Thesis at Toyo University, 2003). This flavone 7-β-glucuronide having an apigenin backbone shows marked copigment effects on the main anthocyanin pigment, and is thus considered to be responsible for the flower color of Veronica persica. The present inventors isolated the flavonoid 7-O-glucuronosyltransferase (F7GAT) gene from cDNA from the petals of Veronica persica using PCR and obtained the polynucleotide (SEQ ID NO: 7) which is one embodiment of the present invention.

### 1. Polynucleotide of the Invention

First, the present invention provides (a) a polynucleotide comprising a polynucleotide consisting of the nucleotide sequence at positions 1 to 1362 in the nucleotide sequence represented by SEQ ID NO: 7 (specifically, a DNA, hereinafter sometimes simply referred to as “DNA”), and (b) a polynucleotide comprising a polynucleotide encoding a protein having the amino acid sequence represented by SEQ ID NO: 8. The DNA targeted in the present invention is not limited only to the DNA encoding the glucuronosyltransferase described above but also includes other DNA encoding a protein functionally equivalent to this protein.

The functionally equivalent protein is, for example, (c) a protein consisting of the amino acid sequence represented by SEQ ID NO: 8 in which 1 to 15 amino acids are deleted, substituted, inserted and/or added and having a UDP-glucuronosyltransferase activity. Such a protein includes, for example, a protein consisting of the amino acid sequence represented by SEQ ID NO: 8 wherein 1 to 15, 1 to 14, 1 to 13, 1 to 12, 1 to 11, 1 to 10, 1 to 9, 1 to 8, 1 to 7, 1 to 6 (1 to several), 1 to 5, 1 to 4, 1 to 3, 1 to 2 or 1 amino acid(s) is/are deleted, substituted, inserted and/or added and having the UDP-glucuronosyltransferase activity. In general, the smaller the number of deletions, substitutions, insertions, and/or additions is, the more preferable it is.

The functionally equivalent protein also includes, for example, (d) a protein having an amino acid sequence having at least 80% homology to the amino acid sequence represented by SEQ ID NO: 8 and having a UDP-glucuronosyltransferase activity. Such a protein includes a protein having an amino acid sequence having a homology of approximately 80% or higher, 81% or higher, 82% or higher, 83% or higher, 84% or higher, 85% or higher, 86% or higher, 87% or higher, 88% or higher, 89% or higher, 90% or higher, 91% or higher, 92% or higher, 93% or higher, 94% or higher, 95% or higher, 96% or higher, 97% or higher, 98% or higher, 99% or higher, 99.1% or higher, 99.2% or higher, 99.3% or higher, 99.4% or higher, 99.5% or higher, 99.6% or higher, 99.7% or higher, 99.8% or higher, or 99.9% or higher, to the amino acid sequence represented by SEQ ID NO: 8, and having the UDP-glucuronosyltransferase activity. In general, the higher the homology percentage above is, the more preferable it is.

As used herein, the term “UDP-glucuronosyltransferase activity” refers to an activity of catalyzing the reaction that involves the glucuronidation of hydroxyl groups of the glycosyl acceptor substrates such as flavonoids, stilbenes, lignans, etc. (glucuronidation of, e.g., a flavonoid at its position 7-OH) to form glucuronide conjugates.

The UDP-glucuronosyltransferase activity can be assayed, for example, by reacting UDP-glucuronic acid with a glycosyl acceptor substrate (e.g., a flavone) in the presence of an analyte enzyme to be assayed and analyzing the reaction product by HPLC, etc. (cf, EXAMPLES later described for more details).

The present invention further includes (e) a polynucleotide comprising a polynucleotide that hybridizes under stringent conditions with a polynucleotide consisting of a nucleotide sequence complementary to the nucleotide sequence at positions 1 to 1362 in the nucleotide sequence represented by SEQ ID NO: 7 and encodes a protein having a UDP-glucuronosyltransferase activity, and (f) a polynucleotide comprising a polynucleotide that hybridizes under stringent conditions with a polynucleotide consisting of a nucleotide sequence complementary to the nucleotide sequence of a polynucleotide encoding a protein consisting of the amino acid sequence represented by SEQ ID NO: 8 and encodes a protein having the UDP-glucuronosyltransferase activity.

As used herein, the “polynucleotide” is used to mean a DNA or RNA, preferably a DNA.

As used herein, the term “polynucleotide that hybridizes under stringent conditions” refers to, for example, a polynucleotide obtained by colony hybridization, plaque hybridization, Southern hybridization or the like, using as a probe all or part of a polynucleotide consisting of a nucleotide sequence complementary to the nucleotide sequence at positions 1 to 1362 in the nucleotide sequence represented by SEQ ID NO: 7 or a polynucleotide consisting of a nucleotide sequence complementary to a nucleotide sequence of a polynucleotide encoding the amino acid sequence represented by SEQ ID NO: 8. The hybridization method may be a method described in, for example, Sambrook & Russell, Molecular Cloning: A Laboratory Manual Vol. 3, Cold Spring Harbor, Laboratory Press 2001, Ausubel, Current Protocols in Molecular Biology, John Wiley & Sons 1987-1997, etc.

As used herein, the “stringent conditions” may be any of low stringent conditions, moderate stringent conditions or high stringent conditions. The “low stringent conditions” are, for example, 5×SSC, 5×Denhardt's solution, 0.5% SDS, 50% formamide and 32° C. The “moderate stringent conditions” are, for example, 5×SSC, 5×Denhardt's solution, 0.5% SDS, 50% formamide and 42° C. The “high stringent conditions” are, for example, 5×SSC, 5×Denhardt's solution, 0.5% SDS, 50% formamide and 50° C. Under these conditions, as the temperature is higher, a DNA with higher homology is expected to be obtained efficiently at higher temperature, although multiple factors are involved in the hybridization stringency including temperature, probe concentration, probe length, ionic strength, time, salt concentration and the like. A person skilled in the art may achieve a similar stringency by appropriately choosing these factors.

When a commercially available kit is used for hybridization, for example, Alkphos Direct Labeling Reagents (manufactured by Amersham Pharmacia) can be used. In this case, according to the attached protocol, a membrane is incubated with a labeled probe overnight, the membrane is washed with a primary wash buffer containing 0.1% (w/v) SDS at 55° C. and the hybridized DNA can then be detected.

In addition to those described above, other polynucleotides that can be hybridized include DNAs having a homology of approximately 60% or higher, approximately 70% or higher, 71% or higher, 72% or higher, 73% or higher, 74% or higher, 75% or higher, 76% or higher, 77% or higher, 78% or higher, 79% or higher, 80% or higher, 81% or higher, 82% or higher, 83% or higher, 84% or higher, 85% or higher, 86% or higher, 87% or higher, 88% or higher, 89% or higher, 90% or higher, 91% or higher, 92% or higher, 93% or higher, 94% or higher, 95% or higher, 96% or higher, 97% or higher, 98% or higher, 99% or higher, 99.1% or higher, 99.2% or higher, 99.3% or higher, 99.4% or higher, 99.5% or higher, 99.6% or higher, 99.7% or higher, 99.8% or higher or 99.9% or higher, to a DNA encoding a DNA of the nucleotide sequence at positions 1 to 1362 in the nucleotide sequence represented by SEQ ID NO: 7, or a DNA encoding the amino acid sequence represented by SEQ ID NO: 8, as calculated by homology search software, such as FASTA and BLAST using default parameters.

Homology between amino acid sequences or nucleotide sequences can be determined by using algorithm BLAST by Karlin and Altschul (Proc. Natl. Acad. Sci. USA, 87: 2264-2268, 1990; Proc. Natl. Acad. Sci. USA, 90: 5873, 1993). Programs called BLASTN and BLASTX based on BLAST algorithm have been developed (Altschul, S. F. et al., J. Mol. Biol. 215: 403, 1990). When a nucleotide sequence is sequenced using BLASTN, the parameters are, for example, score=100 and word length=12. When an amino acid sequence is sequenced using BLASTX, the parameters are, for example, score=50 and word length=3. When BLAST and Gapped BLAST programs are used, default parameters for each of the programs are employed.

The polynucleotide of the present invention described above can be acquired by known genetic engineering techniques or known synthetic techniques.

### 2. Protein of the Invention

In a further embodiment, the present invention also provides the protein encoded by the polynucleotide of the present invention described above. In an embodiment of the present invention, the protein of the present invention is a protein consisting of the amino acid sequence represented by SEQ ID NO: 8. In a further embodiment of the present invention, the protein is a protein having the amino acid sequence represented by SEQ ID NO: 8. In a still further embodiment of the present invention, the protein is a protein consisting of the amino acid sequence represented by SEQ ID NO: 8 in which 1 to 15 amino acids are deleted, substituted, inserted and/or added and having the UDP-glucuronosyltransferase activity. Such a protein includes a protein having the amino acid sequence having the homology described above to the amino acid sequence represented by SEQ ID NO: 8 and having the UDP-glucuronosyltransferase activity. These proteins may be obtained by using site-directed mutagenesis described in Sambrook & Russell, Molecular Cloning: A Laboratory Manual, Vol. 3, Cold Spring Harbor, Laboratory Press 2001, Ausubel, Current Protocols in Molecular Biology, John Wiley & Sons 1987-1997, Nuc. Acids Res., 10, 6487 (1982), Proc. Natl. Acad. Sci. USA, 79, 6409 (1982), Gene, 34, 315 (1985), Nuc. Acids Res., 13, 4431 (1985), Proc. Natl. Acad. Sci. USA, 82, 488 (1985), etc.

In the amino acid sequence for the protein of the present invention, the deletion, substitution, insertion and/or addition of one or more (e.g., 1 to 15, preferably 10 or less) amino acid residues means that one or a plurality of amino acid residues are deleted, substituted, inserted and/or added at one or a plurality of positions in the same amino acid sequence. Two or more types of deletion, substitution, insertion and addition may occur concurrently.

Examples of amino acid residues which are mutually substitutable are given below. Amino acid residues in the same group are mutually substitutable.

Group A: leucine, isoleucine, norleucine, valine, norvaline, alanine, 2-aminobutanoic acid, methionine, o-methylserine, t-butylglycine, t-butylalanine and cyclohexylalanine; Group B: aspartic acid, glutamic acid, isoaspartic acid, isoglutamic acid, 2-aminoadipic acid and 2-aminosuberic acid; Group C: asparagine and glutamine; Group D: lysine, arginine, ornithine, 2,4-diaminobutanoic acid and 2,3-diaminopropionic acid; Group E: proline, 3-hydroxyproline and 4-hydroxyproline; Group F: serine, threonine and homoserine; and Group G: phenylalanine and tyrosine.

The protein of the present invention may also be produced by chemical synthesis methods such as the Fmoc method (fluorenylmethyloxycarbonyl method) and the tBoc method (t-butyloxycarbonyl method). In addition, peptide synthesizers available from Advanced ChemTech, Perkin Elmer, Pharmacia, Protein Technology Instrument, Synthecell-Vega, PerSeptive, Shimadzu Corp., etc. may also be used for the chemical synthesis.

Herein, the protein of the present invention is a glucuronosyltransferase. The term “glucuronosyltransferase” catalyzes the reaction of transferring the glucuronic acid residue from a glycosyl donor to a glycosyl acceptor substrate to form the glucuronide conjugate. In the present invention, the glycosyl acceptor substrate is, for example, a flavonoid, a stilbene, a coumarin and a lignan. The glycosyl donor is, e.g., UDP-glucuronic acid. In an embodiment of the present invention, the protein catalyzes the reaction of transferring the glucuronic acid residue from UDP-glucuronic acid to a glycosyl acceptor substrate to form the glucuronide conjugate and UDP.

The flavonoids which are glycosyl acceptor substrates include flavones, flavonols, flavanones, isoflavones, flavone C-glycosides, aurones, catechins, and the like. Among them, examples of the flavones include baicalein, scutellarein, apigenin, luteolin, tricetin, diosmetin and chrysoeriol. Examples of the flavonols include quercetin, myricetin and kaempferol. An example of the flavanones is naringenin. Examples of the isoflavones are genistein, daidzein and formononetin. Examples of the flavone C-glycosides include vitexin, isovitexin and orientin. An example of the aurones is aureusidin. Examples of the catechins are catechin and epigallocatechin gallate.

The stilbene includes resveratrol and its glycoside piceid, etc.

The lignan includes (+)-pinoresinol, (+)-piperitol, (+)-sesaminol, (+)-secoisolariciresinol, (+)-sesamin catechol 1) (SC 1), (+)-sesamin catechol 2 (SC2), (+)-episesamin catechol 2 (EC2), matairesinol, etc.

In an embodiment of the present invention, the glycosyl acceptor substrate is a flavonoid. In another embodiment of the present invention, the glycosyl acceptor substrate is a flavone with a hydroxy group at the 4′ position of the ring B. In a further embodiment of the present invention, the glycosyl acceptor substrate is at least one glycosyl acceptor substrate selected from the group consisting of scutellarein, apigenin, luteolin, diosmetin, chrysoeriol, kaempferol and naringenin.

For example, the glucuronosyltransferase (VpF7GAT) consisting of the amino acid sequence of SEQ ID NO: 8 shows an activity when the glycosyl acceptor substrate is a flavones such as scutellarein, baicalein, apigenin, luteolin, diosmetin, chrysoeriol, etc., flavonols such as quercetin, kaempferol, etc. and flavanones such as naringenin, etc. Especially when the substrate is scutellarein, apigenin, luteolin, diosmetin, chrysoeriol, kaempferol and naringenin, the activity is strong as compared to other glycosyl acceptor substrates.

### 3. Vector and Transformant Bearing the Same

In another embodiment, the present invention provides the expression vector comprising the polynucleotide of the present invention. The vector of the present invention comprises the polynucleotide of the present invention (e.g., any one of the polynucleotides (a) to (j) described above). Preferably, the expression vector of the present invention comprises any one of the polynucleotides (g) to (j) described above. More preferably, the expression vector of the present invention comprises a polynucleotide consisting of the nucleotide sequence at positions 1 to 1362 in the nucleotide sequence represented by SEQ ID NO: 7, or a polynucleotide comprising a polynucleotide encoding a protein consisting of the amino acid sequence of SEQ ID NO: 8.

The vector of the present invention is generally constructed to contain an expression cassette comprising (i) a promoter that can be transcribed in a host cell,

(ii) the polynucleotide of the present invention linked to the promoter above (e.g., any one of the polynucleotides described in (a) to (j) above), and (iii) a signal that functions in a host cell with respect to the transcription termination and polyadenylation of RNA molecule. The vector thus constructed is introduced into a host cell. To construct the expression vector, methods using a plasmid, phage or cosmid are used but are not particularly limited.

Specific types of the vector are not particularly limited, and vectors capable of expressing in a host cell can be suitably chosen. That is, a suitable promoter sequence may be chosen depending upon the type of the host cell to reliably express the polynucleotide of the present invention, and a vector obtained by incorporating this sequence and the polynucleotide of the present invention into various plasmids or the like may be used as an expression vector.

The expression vector of the present invention contains an expression control region (for example, a promoter, a terminator, and/or a replication origin, etc.) depending on the type of a host to be introduced. A conventional promoter (for example, trc promoter, tac promoter, lac promoter, etc.) is used as a promoter for a bacterial expression vector. As a promoter for yeast, there are used, for example, a glyceraldehyde 3-phosphate dehydrogenase promoter, PH05 promoter, etc. As a promoter for fungi there are used, for example, amylase, trpC, etc. Additionally, a viral promoter (e.g., SV40 early promoter, SV40 late promoter, etc.) is used as a promoter for animal-derived host cell.

The expression vector preferably contains at least one selective marker. The marker available includes an auxotrophic marker (ura5, niaD), a drug-resistant marker (hygromycin, zeocin), a geneticin-resistant marker (G418r), a copper-resistant gene (CUP1) (Marin et al., Proc. Natl. Acad. Sci. USA, 81, 337, 1984), a cerulenin resistant gene (fas2m, PDR4) (Junji Inokoshi et al., Biochemistry, 64, 660, 1992; and Hussain et al., Gene, 101: 149, 1991, respectively), and the like.

The present invention provides the transformant in which the polynucleotide of the present invention (for example, the polynucleotide described in any one of (a) to (j) above) is introduced.

A method of preparing (method of producing) the transformant is not particularly limited and includes, for example, a method which comprises introducing the recombinant vector into a host followed by transformation. The host cell used herein is not particularly limited and various known cells may be preferably used. Specific examples are bacteria such as Escherichia coli, etc., yeast (budding yeast Saccharomyces cerevisiae, fission yeast Schizosaccharomyces pombe), nematode (Caenorhabditis elegans), oocyte of African clawed frog (Xenopus laevis), etc. Culture media and conditions suitable for the host cells above are well known in the art. The organism to be transformed is not particularly limited, and includes various microorganisms, plants and animals given as examples of the host cells above.

For transformation of the host cell, there may be used generally known methods. For example, methods that can be used include but not limited to the electroporation method (Mackenzie D. A. et al., Appl. Environ. Microbiol., 66, 4655-4661, 2000), the particle delivery method (method described in JPA 2005-287403 “Method of Breeding Lipid-Producing Fungus”), the spheroplast method (Proc. Natl. Acad. Sd. USA, 75: 1929 (1978)), the lithium acetate method (J. Bacteriology, 153: 163 (1983)), and methods described in Proc. Natl. Acad. Sci. USA, 75: 1929 (1978), Methods in yeast genetics, 2000 Edition: A Cold Spring Harbor Laboratory Course Manual, etc.

In another embodiment of the present invention, the transformant can be a plant transformant. The plant transformant in accordance with the present invention can be acquired by introducing a recombinant vector bearing the polynucleotide of the present invention into a plant in such a manner that the polypeptide encoded by the said polynucleotide can be expressed.

Where a recombinant expression vector is used, the recombinant expression vector used to transform the plant is not particularly limited as far as the vector is capable of expressing the polynucleotide of the present invention in said plant. Examples of such vectors include a vector bearing a promoter capable of constitutively expressing the polynucleotide in plant cells (e.g., a 35S promoter of cauliflower mosaic virus), and a vector inducibly activated by external stimulation.

Plants which are to be the target of transformation in the present invention may be any of entire plant bodies, plant organs (e.g., leaves, petals, stems, roots, seeds, etc.), plant tissues (e.g., epidermis, phloem, parenchyma, xylem, vascular bundles, palisade tissues, spongy tissues, etc.) or plant culture cells, or various types of plant cells (e.g., suspension culture cells), protoplasts, leaf slices, calli, and the like. Specific examples of plant species which are used for transformation include, but are not limited to, those belonging to the Monocotyledoneae or the Dicotyledoneae.

For transformation of genes into plants, conventional transformation methods known to one skilled in the art (e.g., the Agrobacterium method, gene gun, the PEG method, the electroporation method, etc.) are used. For example, the Agrobacterium-mediated method and the method of directly introducing into plant cells are well known. When the Agrobacterium method is used, the constructed plant expression vector is introduced into an appropriate Agrobacterium strain (e.g., Agrobacterium tumefaciens), followed by infection of aseptically cultured leaf discs with this strain according to the leaf disc method (Hirobumi Uchimiya, Manuals for Plant Gene Manipulation (1990), 27-31, Kodansha Scientific Co., Ltd., Tokyo). Thus, the transgenic plant can be obtained. In addition, the method of Nagel, et al. (Micribiol. Lett., 67, 325 (1990)) may be used. This method involves introducing first, e.g., an expression vector into Agrobacterium and then introducing the transformed Agrobacterium into plant cells or plant tissues according to the method described in Plant Molecular Biology Manual (S. B. Gelvin, et. al., Academic Press Publishers). Herein, the “plant tissue” includes callus, which is obtained by culturing plant cells. When the transformation is carried out using the Agrobacterium method, binary vectors (pBI121, pPZP202, etc.) can be used.

For direct transfer of genes into plant cells or plant tissues, the electroporation method and the gene gun method are known. When the gene gun is used, entire plant bodies, plant organs or plant tissues per se may be used, or may be used after preparation of sections, or may be used after preparation of protoplasts. The samples thus prepared can be bombarded using a gene transfer apparatus (e.g., PDS-1000 (BIO-RAD, Inc.), etc.). Bombardment conditions vary depending upon type of the plant or sample. Normally, the sample is bombarded under a pressure of about 450 to 2000 psi at a distance of about 4 to 12 cm.

The cells or plant tissues in which the gene is introduced are first selected by chemical resistance such as a hygromycin resistance, etc. and then regenerated into plant bodies in a conventional manner. Regeneration of plant bodies from the transformant cells can be performed by methods known to one skilled in the art, depending upon kind of plant cells.

When a plant culture cell is used as a host, transformation is performed by introducing the recombinant vector into the culture cell by means of gene gun, the electroporation method, etc. Calli, shoots, hairy roots or the like resulting from transformation can be used for cell culture, tissue culture or organ culture as they are. Alternatively, these tissues or cells can be allowed to regenerate into a plant by any known conventional method for culturing plant tissues by administering a plant hormone (auxin, cytokinin, gibberellin, abscisic acid, ethylene, brassinolide, etc.).

Whether or not the gene is introduced into a plant can be confirmed by PCR, the Southern hybridization, the northern hybridization or the like. For example, DNA is prepared from transformed plants, DNA-specific primers are then designed, and PCR is subsequently performed. PCR can be performed under the same conditions as used for preparing the plasmids described above. Then, the transformation can be confirmed by applying the amplified product to agarose gel electrophoresis, polyacrylamide gel electrophoresis, capillary electrophoresis or the like, staining the product with an appropriate dye, such as ethidium bromide, SYBR Green, etc. and then detecting the amplified product as a single band. In addition, amplified products can also be detected by performing PCR using primers labeled with a suitable label, e.g., a fluorescent dye. Furthermore, other methods that can also be employed herein involve binding the amplified product to a solid phase, such as a microplate, and then confirming the amplified product by fluorescence, an enzyme reaction or the like.

Once a transformed plant wherein the polynucleotide of the present invention is introduced into the genome is obtained, it is possible to acquire descendants from that plant body by sexual or asexual reproduction. Alternatively, plants can be mass-produced from breeding materials (for example, seeds, fruits, ears, tubers, tubercles, tubs, calli, protoplast, etc.) obtained from the plant, as well as descendants or clones thereof. Transformed plant bodies capable of expressing the polynucleotide of the present invention, descendants of the plant bodies which have the same qualities as the plant bodies, as well as tissues obtained from the plant bodies or the descendants, are all included in the present invention.

In addition, methods for transformation of various plants have already been reported. Examples of the transformant plant in accordance with the present invention include, but are not limited to, sesame, rice plant, tobacco, barley, wheat, rapeseed, potato, tomato, poplar, banana, eucalyptus, sweet potato, soybean, alfalfa, lupin, corn, cauliflower, rose, chrysanthemum, carnation, snapdragon, cyclamen, orchid, lisianthus, freesia, gerbera, gladiolus, soaproot, kalanchoe, lily, pelargonium, geranium, petunia, torenia, tulip, Forsythia, Arabidopsis, Lotus, etc.

In an embodiment of the present invention, the transformed plant is a plant for functional food materials.

In another embodiment of the present invention, the transformed plant is a plant with a modified flower color. Preferably, the plant with a modified flower color is a plant with its flower color being modified to a blue color.

### 4. Method of Producing the Protein of the Invention

In yet another embodiment, the present invention provides a method of producing the protein of the present invention using the transformants described above.

Specifically, the protein of the present invention may be obtained by isolating and purifying the protein of the present invention from the culture of the transformants described above. As used herein, the culture refers to any one of a culture broth, cultured bacteria or cultured cells, and the homogenate of cultured bacteria or cultured cells. Conventional methods may be used to isolate and purify the protein of the present invention.

Specifically, when the protein of the present invention accumulates within cultured bacteria or within cultured cells, a crude extract of the protein of the present invention may be obtained by culturing the bacteria or cells, then disrupting the bacterial or cells using a conventional technique (e.g., ultrasonication, lysozymes, freezing and thawing, etc.) and applying a conventional method such as centrifugation or filtration. When the protein of the present invention is accumulated in the culture broth, the culture supernatant containing the protein of the present invention can be obtained, after completion of the incubation, by separating the bacteria or cells from the culture supernatant in a conventional manner (e.g., centrifugation, filtration, etc.).

Purification of the protein of the present invention contained in the extract or culture supernatant obtained as described above can be performed by a conventional method of separation and purification. The separation and purification methods including ammonium sulfate precipitation, gel filtration chromatography, ion exchange chromatography, affinity chromatography, reversed phase high performance liquid chromatography, dialysis, and ultrafiltration, etc. may be used singly or in a suitable combination.

### 5. Method of Producing Glucuronide Conjugates

The present invention further provides a method of producing the glucuronide conjugate using the protein of the present invention. The protein of the present invention catalyzes the reaction of transferring the glucuronic acid from the glycosyl donor (e.g., UDP-glucuronic acid) to the glycosyl acceptor substrate (e.g., a flavonoid, a stilbene or a lignan). Therefore, the glucuronide conjugate can be produced from the glycosyl acceptor substrate and the glycosyl donor as the starting materials by using the protein of the present invention. The glycosyl acceptor substrate is preferably a flavonoid.

The glucuronide can be produced, for example, by preparing a solution containing 1 mM of the glycosyl acceptor substrate, 2 mM of the glycosyl donor, 50 mM of calcium phosphate buffer (pH 7.5) and 20 μM of the protein of the present invention and reacting them at 30° C. for 30 minutes. The glucuronide conjugate can be isolated/purified from the solution by known methods. Specifically, e.g., ammonium sulfate precipitation, gel filtration chromatography, ion exchange chromatography, affinity chromatography, reversed phase high performance liquid chromatography, dialysis, ultrafiltration, etc. can be used alone or in an appropriate combination.

The glucuronide conjugate thus obtained is useful as a reagent for functional food materials, for inspecting their in vivo functions, or as an antioxidant, etc. (Gao, Z., Huang, K., Yang, X., and Xu, H. (1999) Biochimica et Biophysica Acta, 1472, 643-650).

### EXAMPLES

The present invention is described in more details with reference to EXAMPLES below but is not deemed to be limited thereto.

### Example 1

### Gene Cloning

The molecular biological procedures used in this EXAMPLE were performed in accordance with the methods described in Molecular Cloning (Sambrook et al., Cold Spring Harbour Laboratory Press, 2001), unless indicated elsewhere in detail.

It was found by homology search using the BLAST analysis that the glucosyltransferase gene (AmUGTcgl 0, Accession No. AB362988) for Scrophulariaceae Antirrhinum majus had 55% sequence homology on the amino acid sequence level to the glucosyltransferase gene SbUBGAT (Nagashima S. et al., Phytochemistry 53, 533-538, 2000) for Lamiaceae Scutellaria baicalensis (Ono, E. et al., Proc. Natl. Acad. Sci. USA 103, 11075-11080, 2006).

To isolate the gene encoding the flavonoid 7-O-glucuronosyltransferase VpF7GAT of Veronica persica of the same genus Scrophulariaceae, the two primers (SEQ ID NOS: 1 and 2) shown below were designed based on the sequence of Antirrhinum majus in the same family.

### SEQ ID NO: 1

AmF7GAT-F1: 5′-GTG ATA GAT TTC TTT TGC AAT-3′

### SEQ ID NO: 2

AmF7GAT-R3: 5′-ACC CTA TTC ATC CTC TGC TCC-3′

After total RNA was extracted from the petals of Veronica persica using RNeasy Plant Mini Kit (QIAGEN), cDNA was synthesized from 1 μg of the total RNA using SuperScript First-Strand Synthesis System for RT-PCR (Invitrogen) according to the protocol recommended by the manufacturer. Using the resulting cDNA as a template, PCR was performed using the primers of SEQ ID NOS: 1 and 2 described above to attempt isolation of the gene encoding VpF7GAT.

Specifically, the PCR reaction solution (50 μl) was composed of 1 μl of cDNA from Veronica persica, 1× ExTaq buffer (Takara-Bio), 0.2 mM dNTPs, 0.4 pmol each/μl of the primers (SEQ ID NOS: 1 and 2) and 2.5 U ExTaq polymerase. PCR was performed by reacting at 94° C. for 3 minutes and then amplifying for 35 cycles with each cycle at 94° C. for 1 minute, 50° C. for 1 minute and 72° C. for 2 minutes.

The PCR solution was separated by 0.8% agarose gel electrophoresis to give the amplification fragment corresponding to the size of about 1.0 kb. The amplified fragment was inserted into the multicloning site of pCR-TOPOII vector (Invitrogen). The nucleotide sequence of the inserted fragment was sequenced by the primer walking method using synthetic oligonucleotide primers with a DNA Sequencer Model 3100 (Applied Biosystems). The nucleotide sequence determined was analyzed using the CLUSTAL-W Program (MACVECTOR 7.2.2 Software, Accerly) and as a result, showed a high sequence homology to Antirrhinum majus AmUGTcg10 and Scutellaria baicalensis SbUBGAT. This cDNA was thus identified as a candidate gene for VpF7GAT.

However, the results of alignment with AmUGTcg10 shows that the cDNA amplified fragment obtained was an incomplete ORF (open reading frame) deleted of the 5′ and 3′ regions. Accordingly, rapid amplification of cDNA end (hereinafter abbreviated as RACE) was performed using the Gene Racer Kit (Invitrogen) according to the method recommended by the manufacturer to amplify the 5′ and 3′ regions of the cDNA fragment. For RACE, there were used a set of the primers specific to the VpF7GAT gene, which are shown by SEQ ID NOS: 3 to 6 below.

SEQ ID NO: 3
GR-VpF7GAT-RV:
5′-TTC CAG GAG GGT TTC GAA CGG ACC ATA-3′
SEQ ID NO: 4
GR-VpF7GAT-nest-RV:
5′-CTA GAG GTG CAA CGA ATA AAA CTT-3′
SEQ ID NO: 5
GR-VpF7GAT-Fw:
5′-TAT GGT CCG TTC GAA ACC CTC CTG GAA-3′
SEQ ID NO: 6
GR-VpF7GAT-nest-Fw:
5′-AGG ATC CTG ACC TGG AAA CA-3′

The nucleotide sequence of the amplified fragment obtained by RACE was determined by the primer walking method to give a candidate gene for VpF7GAT containing the full length ORF and its amino acid sequence (SEQ ID NO: 7 (cDNA sequence of VpF7GAT), SEQ ID NO: 8 (amino acid sequence of VpF7GAT)).

This VpF7GAT candidate gene showed 61% and 51% sequence homologies, respectively, to Antirrhinum majus AmUGTcg 10 and Scutellaria baicalensis SbUBGAT, on the amino acid sequence level.

### Example 2

### Construction of Vector

To clarify biological functions of the candidate protein for VpF7GAT obtained in EXAMPLE 1 (hereinafter this enzyme), an Escherichia coli expression vector capable of expressing cDNA for this enzyme was constructed. cDNA containing the full length ORF was amplified by PCR using a set of the primers represented by SEQ ID NOS: 9 and 10, specific to the candidate gene for VpF7GAT. As a template, cDNA synthesized using the total RNA extracted from the petals of Veronica persica described above was used.

SEQ ID NO: 9
CACC-NdeI-VpF7GAT-Fw:
5′-CAC CCA TAT GGA AGA CAC AAT CAT CCT-3′
SEQ ID NO: 10
XhoI-VpF7GAT-Rv:
5′-CTC GAG TTT TTA CCC AAT AAC CAA CTT GAT-3′

PCR (KOD Plus Polymerase, TOYOBO) was performed, after thermal denaturation at 94° C. for 2 mins., with [94° C. for 15 secs., 50° C. for 30 secs. and 68° C. for 1.5 mins.]×35 cycles. The amplified DNA fragment was subcloned to pCR-Blunt II-TOPO vector (Zero Blunt TOPO PCR Cloning Kit, Invitrogen). The nucleotide sequence was confirmed by ABI 3100 Avant Genetic Analyzer (Applied Biosystems).

The plasmid obtained was fully digested with restriction enzymes NdeI and XhoI, and the resulting DNA fragment of about 1.5 kb containing the full length ORF was ligated to the E. coli expression vector pET-15b (Novagen) at the NdeI and XhoI sites to give the E. coli expression vector.

### Example 3

### Expression and Purification of Escherichia Coli Recombinant Protein

Using the respective plasmids obtained above, the E. coli BL21 (DE3) strain was transformed in a conventional manner. The transformant obtained was shake cultured in 4 ml of LB medium (10 g/l typtone pepton, 5 g/l yeast extract, 1 g/l NaCl) containing 50 μg/ml of ampicillin at 37° C. overnight. When the cells reached the stationary phase, 4 ml of the culture broth was inoculated into 80 ml of a medium of the same composition, followed by shake culture at 37° C. At the point when the cell turbidity (OD 600) became approximately 0.7, IPTD was added to the cells in a final concentration of 0.5 mM, followed by shake culture at 22° C. for 20 hours.

The following procedures were all performed at 4° C. The transformant cultured was collected by centrifugation (7,000×g, 15 mins.) and 2 ml/g cell of Buffer S [20 mM sodium phosphate buffer (pH 7.4), 20 mM imidazole, 0.5 M NaCl, 14 mM β-mercaptoethanol] was added to suspend the cells. Subsequently, ultrasonication was performed (15 secs.×8 times), followed by centrifugation (15,000×g, 10 mins.). Polyethylenimine was added to the supernatant obtained in a final concentration of 0.12% (w/v) and the resulting suspension was allowed to stand for 30 minutes. After centrifugation (15,000×g, 10 mins.), the supernatant was recovered as a crude enzyme solution. The crude enzyme solution was applied to His SpinTrap (GE Healthcare) equilibrated with Buffer S and then centrifuged (70×g, 30 secs.). After washing with 600 μl of Buffer S, the protein bound to the column was stepwise eluted with 600 μl each of Buffer S containing 100, 200 and 500 mM imidazole. In each of the eluted fractions, the buffer was replaced with 20 mM potassium phosphate buffer (pH 7.5) containing 14 mM β-mercaptoethanol using a Microcon YM-30 (Amicon).

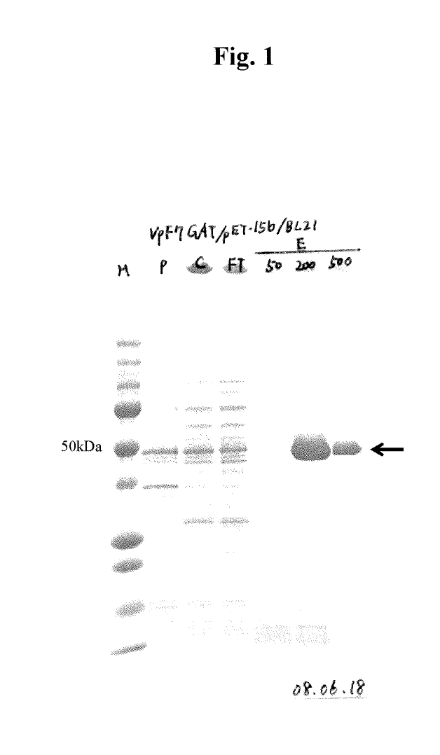

As a result of the SDS-PAGE analysis, the expressed protein purified at about 50 kDa deduced from the amino acid sequence of VpF7GAT was confirmed in the fraction eluted with 200 mM imidazole. Thus, this fraction was used for enzyme analysis (FIG. 1, arrow: target protein).

### Example 4

### Enzyme Reaction and Product Analysis

Standard reaction conditions are as follows. After 50 μl of the reaction solution (2 mM UDP-glucuronic acid, 100 μM glycosyl acceptor substrate, 50 mM potassium phosphate buffer (pH 7.5), enzyme solution) was prepared, the enzyme solution was added thereto to initiate the reaction. The mixture was reacted at 30° C. for 1 minute. The reaction was then terminated by adding 50 μl of CH₃CN containing 0.5% TFA. The product was analyzed by reversed phase HPLC (LC-2010 System, Shimadzu Corporation).

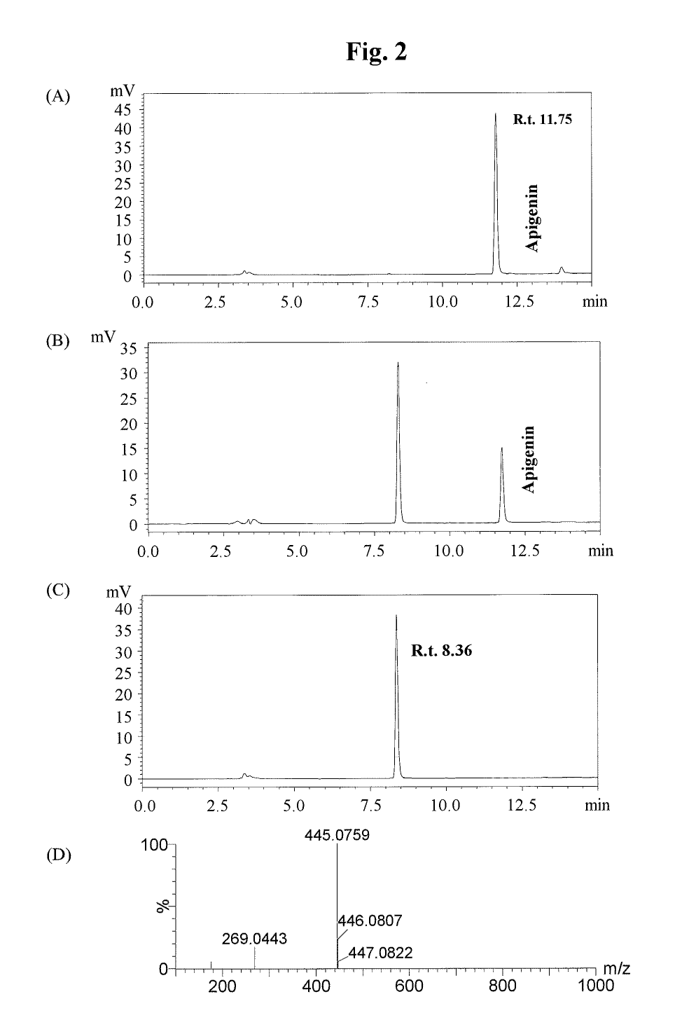

The conditions for HPLC are as follows. Develosil C30-UG-5 column (4.6 mm×150 mm, Nomura Chemical) was used with a column oven at 40° C., and the moving phase A (0.1% TFA/H₂O) and the moving phase B (0.1% TFA/90% CH₃CN) were used. The conditions for elution include a linear density gradient (B20%→B70%) for 15 minutes, then retention with B70% for further 1 minute and again reverting to B20%, followed by equilibration for 20 minutes. HPLC was performed at the flow of 1 ml/min. Detection was performed at 280 and 360 nm using a SPD-M10A Photodiode Array Detector (Shimadzu Corporation). Under the conditions, apigenin (Funakoshi) as a standard, apigenin 7-O-glucuronide purified from the petals of Antirrhinum majus and apigenin 7-O-glucoside (Funakoshi) were eluted at retention times of approximately 11.75 minutes, 8.36 minutes and 8.22 minutes, respectively (FIG. 2(A): apigenin, FIG. 2(C): apigenin 7-β-glucuronide).

The enzyme reaction solution obtained using apigenin as the glycosyl acceptor substrate and UDP-glucuronic acid as the glycosyl donor was analyzed by HPLC. As a result, the formation of a novel product having a retention time coincident with apigenin 7-O-glucuronide used as the standard was confirmed (FIG. 2(B)).

The conditions for LC-MS are as follows. Column used: Develosil C30-UG-3 column (Nomura Chemical, 3.0 mm×150 mm); Moving phase used: water containing 0.1% formic acid as eluant A and as eluant B 100% acetonitrile containing 0.1% formic acid. Elution was performed using a linear density gradient (eluant B: 20%→70%) for 20 minutes, followed by isocratic elution with 70% eluant B for 5 minutes (flow rate: 0.2 ml/min., column oven: 40° C.).

Detection was achieved by collecting the data at 230-500 nm and monitoring the chromatogram at A337 nm using a photodiode array detector (SPD-M10A, Shimadzu Corporation). A TOF-MS detector (Q-TOF Premier, Micromass, UK) was connected to the PDA detector at the back. The molecular weight of the product was determined under the following conditions. MS was determined under the measurement conditions in an negative mode (ion source: ESI, lock spray reference: leucine encephalin (m/z 554.2615 [M-H]⁻), capillary: 2.7 kV, cone: 30 V, MS/MS collision energy: 20 eV).

Under the conditions, apigenin which is the substrate eluted at the retention time of 17.51 minutes gave the molecular ions of m/z 269.0441 [M-H]⁻. On the other hand, the product eluted at the retention time of 11.72 minutes gave the molecular ions of m/z 445.0759 [M-H]⁻, and was identified to have one glucuronic acid attached to apigenin (FIG. 2(D)). Further by MS/MS analysis, the fragment ions of m/z 269.0450 [M-H]⁻ coincident with apigenin was detected from this product.

The foregoing results revealed that this enzyme was found to be the protein having the F7GAT activity of Veronica persica.

### Example 5

### Analysis of Enzyme Functions of VpF7GAT

Selectivity of this enzyme to UDP glycosyl donors (glycosyl acceptor substrate: apigenin) was determined in a manner similar to the method of Noguchi, A. et al., Plant J. 54, 415-427, 2008. When the activity of UDP-glucuronic acid was made 100%, the relative activity of UDP-glucose was 5.8% and that of UDP-galactose was the level below the detection limit. The high specificity of this enzyme to UDP-glucuronic acid was thus confirmed. Furthermore, the substrate specificities (Km) of this enzyme to apigenin and UDP-glucuronic acid were 10.7±1.7 μM and 36.6±8.7 respectively, and the catalytic activity (kcat) for apigenin was 8.64 S⁻¹.

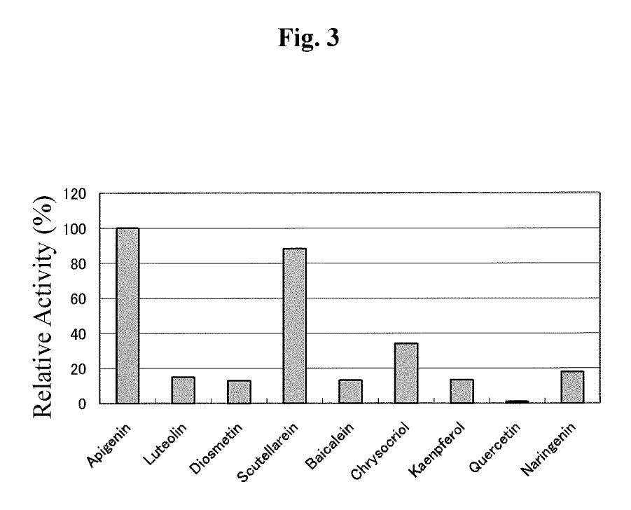

Selectivity of this enzyme to a glycosyl acceptor substrate (glycosyl donor: UDP-glucuronic acid) was examined. This enzyme showed the highest activity to the endogenous substrate apigenin (FIG. 3). When the activity to this apigenin was made 100%, the relative activities of scutellarein, baicalein, luteolin, diosmetin and chrysoeriol which are flavones; quercetin and kaempferol which are flavonols; and naringenin which is a flavanone were 88.3%, 13.1%, 14.9%, 12.9%, 34.0%, 1.0%, 13.3% and 18.0%, respectively.

Especially in diosmetin in which the 4′-hydroxy in the flavonoid B ring is methylated and in the flavonoids having 2 or more hydroxy groups in the B ring, the activity of this enzyme was lower than those having a single hydroxy group at the 4′ position. In addition, since the activity to baicalein having no hydroxy group in the ring B was low, the 4′-hydroxy group in the flavonoid B ring was round to be extremely important for recognition of the glycosyl acceptor substrate.

Also under the reaction conditions for this enzyme, the glucuronosyltransferase activity was not observed for resveratrol which is stilbenes, esculetin which is coumarins, sesaminol which is lignans, daidzein and genistein which are isoflavones, and isovitexin and orientin which are flavone C-glucosides. Among flavonoids, this enzyme was shown to have a strong activity especially on the flavones having the hydroxy group at the 4′ position in the ring B.

Flavones: apigenin, luteolin, diosmetin, chrysoeriol, baicalein and scutellarein Flavone C-glucosides: isovitexin and orientin

In the following formulae, Glc represents glucose.

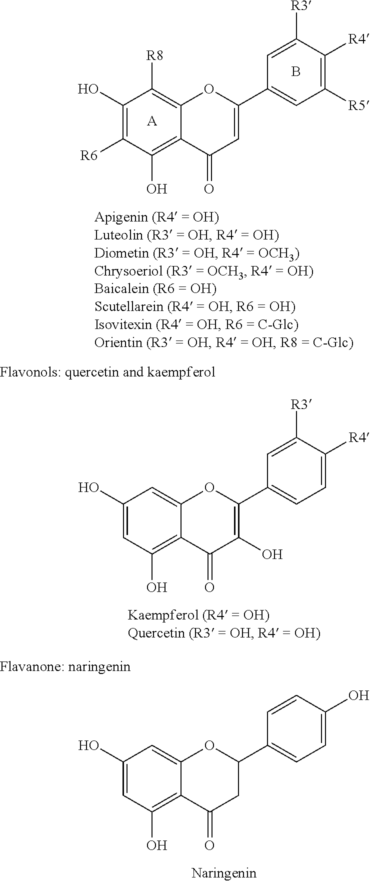

### Example 6

### Expression Analysis of the VpF7GAT Gene

The expression pattern of the VpF7GAT gene in different organs was analyzed by quantitative RT-PCR using 7500 Real Time PCR System (Applied Biosystems) in a manner similar to the method of Noguchi, A. et al., Plant J. 54, 415-427, 2008. The total RNA was extracted from the respective organs (leaves, flowers, fruits, stems and roots) of Veronica persica in a manner similar to Example 1. Then, 1 m of each organ was subjected to reverse transcription (RT) to give cDNA of each organ. This cDNA was used as the template for PCR.

The following 4 primers were designed as the primers specific to each gene used for the quantitative PCR, using a Primer Express 3.0 Program (Applied Biosystems). The VpF7GAT-specific primers used were those of SEQ ID NOS: 11 and 12. Ribosome RNA (AF509785) of Veronica persica was adopted as an internal standard gene. Amplification was effected by using the gene-specific primers of SEQ ID NOS: 13 and 14 shown below.

SEQ ID NO: 11
qVpF7GAT-Fw:
5′-GCG GTT TCG GCC TCT GT-3′
SEQ ID NO: 12
qVpF7GAT-Rv:
5′-TCC GAT ATC TTG AGG GAT GAT TTC-3′
SEQ ID NO: 13
qVprRNA-Fw:
5′-GCG GAA GGA TCA TTG TCG AT-3′
SEQ ID NO: 14
qVprRNA-Rv:
5′-CTA GCG GGC GGA GCT TAT TA-3′

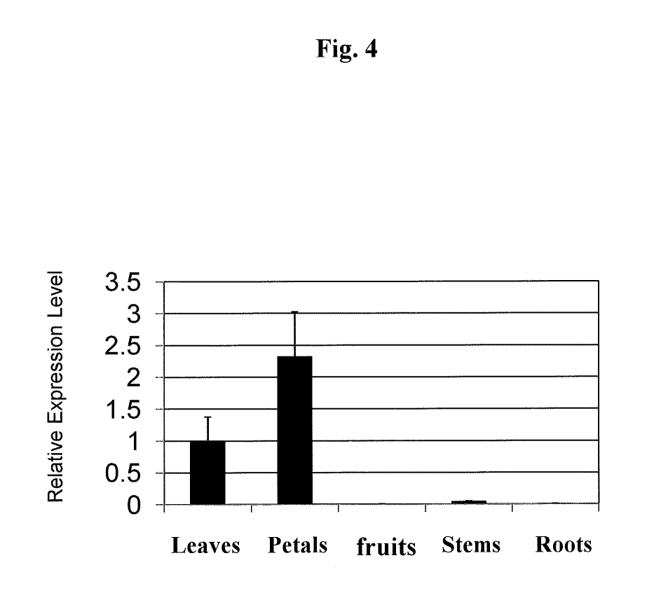

The expression level of VpF7GAT was standardized by the expression level of the internal standard gene. The relative expression level was determined by the ΔΔCt method (Applied Biosystems). The results revealed that the VpF7GAT gene was highly expressed in the petals (FIG. 4). Since the site of flavone 7-O-glucuronic acid accumulation given by this enzyme coincided with the region where the gene for this enzyme was expressed, it was strongly suggested that this enzyme would be involved in developing the flower color of Veronica persica through the formation of a copigment. In addition, no remarkable color development was observed even though the expression of VpF7GAT was recognized also in the leaves. This is considered to be because the amount of major color pigment anthocyanin is extremely low as compared to the amount in the petals.

As described above, the enzyme (VpF7GAT) that can transfer glucuronic acid to the 7-O-position of flavonoids could be isolated from Veronica persica. By using this enzyme, plants with modified flower colors (e.g., blue flowers) can be produced and functional food materials can be developed.

### INDUSTRIAL APPLICABILITY

The UDP-glucuronosyltransferase of the present invention has a broader substrate specificity and is useful for the production of various glucuronide conjugates. By using the glucuronosyltransferase of the present invention, for example, plants with modified flower colors (e.g., blue flowers) or functional food materials can be developed.

1. A polynucleotide of any one of (a) to (f):
(a) a polynucleotide comprising a polynucleotide consisting of the nucleotide sequence at positions 1 to 1362 in the nucleotide sequence represented by SEQ ID NO: 7;
(b) a polynucleotide comprising a polynucleotide encoding a protein having the amino acid sequence represented by SEQ ID NO: 8;
(c) a polynucleotide comprising a polynucleotide encoding a protein consisting of the amino acid sequence represented by SEQ ID NO: 8 in which 1 to 15 amino acids are deleted, substituted, inserted and/or added and having a UDP-glucuronosyltransferase activity;
(d) a polynucleotide comprising a polynucleotide encoding a protein having an amino acid sequence having at least 80% homology to the amino acid sequence represented by SEQ ID NO: 8 and having a UDP-glucuronosyltransferase activity;
(e) a polynucleotide comprising a polynucleotide that hybridizes under stringent conditions with a polynucleotide consisting of a nucleotide sequence complementary to the nucleotide sequence at positions 1 to 1362 in the nucleotide sequence represented by SEQ ID NO: 7 and encodes a protein having a UDP-glucuronosyltransferase activity; and,
(f) a polynucleotide comprising a polynucleotide that hybridizes under stringent conditions with a polynucleotide consisting of a nucleotide sequence complementary to a nucleotide sequence of a polynucleotide encoding a protein consisting of the amino acid sequence represented by SEQ ID NO: 8, and encodes a protein having a UDP-glucuronosyltransferase activity.

2. The polynucleotide according to claim 1, which is any one of (g) to (j) below:
(g) a polynucleotide comprising a polynucleotide encoding a protein consisting of the amino acid sequence represented by SEQ ID NO: 8 in which at most 10 amino acids are deleted, substituted, inserted and/or added and having a UDP-glucuronosyltransferase activity;
(h) a polynucleotide comprising a polynucleotide encoding a protein having an amino acid sequence having at least 90% homology to the amino acid sequence represented by SEQ ID NO: 8 and having a UDP-glucuronosyltransferase activity;
(i) a polynucleotide comprising a polynucleotide that hybridizes under high stringent conditions with a polynucleotide consisting of a nucleotide sequence complementary to the nucleotide sequence at positions 1 to 1362 in the nucleotide sequence represented by SEQ ID NO: 7 and encodes a protein having a UDP-glucuronosyltransferase activity; and,
(j) a polynucleotide comprising a polynucleotide that hybridizes under high stringent conditions with a polynucleotide consisting of a nucleotide sequence complementary to a nucleotide sequence of a polynucleotide encoding a protein consisting of the amino acid sequence represented by SEQ ID NO: 8 and encodes a protein having a UDP-glucuronosyltransferase activity.

3. The polynucleotide according to claim 1, which comprises a polynucleotide consisting of the nucleotide sequence at positions 1 to 1362 in the nucleotide sequence represented by SEQ ID NO: 7.

4. The polynucleotide according to claim 1, which comprises a polynucleotide encoding a protein consisting of the amino acid sequence represented by SEQ ID NO: 8.

5. The polynucleotide according to claim 1, which is a DNA.

6. A protein encoded by the polynucleotide according to claim 1.

7. A vector comprising the polynucleotide according to claim 1.

8. A transformant transformed with the polynucleotide according to claim 1.

9. A transformant transformed with the vector according to claim 7.

10. A method for producing the protein of claim 6, which comprises using the transformant according to claim 8.

11. A method for producing a glucuronide conjugate, which comprises forming the glucuronide conjugate from UDP-glucuronic acid and a glycosyl acceptor substrate using the protein according to claim 6 as a catalyst.
